(19) United States
(12) Patent Application Publication
(10) Pub. No.: US 2009/0298843 A1
(43) Pub. Date: Dec. 3, 2009

(54) TREATMENT OF HEMATOLOGICAL MALIGNANCIES WITH FTS AND A BCR-ABL TYROSINE KINASE INHIBITOR

(75) Inventors: Yoel Kloog, Herzliya (IL); Itay Nakdimon, Ramat Gan (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RAMOT AT TEL AVIV UNIVERSITY LTD., Tel Aviv (IL)

(21) Appl. No.: 12/225,944

(22) PCT Filed: Apr. 10, 2007
(86) PCT No.: PCT/IL2007/000439

§ 371 Date: Jun. 29, 2009

Related U.S. Application Data
(60) Provisional application No. 60/791,050, filed on Apr. 11, 2006.

(51) Int. Cl. A61K 31/506; A61K 31/192

(52) U.S. Cl. 514/252.18; 514/568

(57) ABSTRACT

Disclosed are methods of treating a hematological malignancy by administering to a human in need thereof effective amounts of FTS (Farnesylthiosalicylic Acid), or various analogs thereof, or a pharmaceutically acceptable salt thereof, optionally in combination with a Bcr-Abl tyrosine kinase inhibitor. Also disclosed are pharmaceutical compositions comprising FTS, or various analogs thereof, or a pharmaceutically acceptable salt thereof, a Bcr-Abl tyrosine kinase inhibitor, preferably Imatinib and a pharmaceutically acceptable carrier.

### CROSS-REFERENCE TO RELATED APPLICATIONS

This application claims the benefit of the filing date of U.S. Provisional Patent Application No. 60/791,050, filed Apr. 11, 2006, the disclosure of which is hereby incorporated herein by reference.

### BACKGROUND OF THE INVENTION

Hematological malignancies are cancers that affect blood, bone marrow and lymph nodes. Chromosomal translocations are a common cause of these diseases. For example, a translocation involving chromosomes 9 and 22 is characteristic of the Philadelphia chromosome abnormality (also known as the Bcr-Abl fusion transcript) found in chronic myeloid leukemia. Lymphoma often spreads to the bone marrow, affecting the blood. Thus, diagnosis and treatment of hematological malignancies require a different approach than treatment of solid tumors.

The very nature of hematological malignancies necessitates using systemic chemotherapy as the primary treatment modality. Radiation therapy may be used as an adjunct to treat local accumulations of these cells. Surgery is rarely indicated as a primary treatment modality, but may be used in managing some complications. Bone marrow transplantation from an HLA-matched sibling is sometimes indicated.

### SUMMARY OF THE INVENTION

A first aspect of the present invention is directed to a method of treating a hematological malignancy. The method comprises administering to a human in need thereof an effective amount of farnesylthiosalicylic acid (FTS) or an analog thereof, or a pharmaceutically acceptable salt thereof.

Another aspect of the present invention is directed to a method of treating a hematological malignancy which comprises administering to a human in need thereof effective amounts of farnesylthiosalicylic acid (FTS) or an analog thereof, or a pharmaceutically-acceptable salt thereof, and a Bcr-Abl tyrosine kinase inhibitor. In some embodiments, the Bcr-Abl tyrosine kinase inhibitor is imatinib or a derivative thereof, or a pharmaceutically acceptable salt thereof.

A further aspect of the present invention is directed to a pharmaceutical composition useful in the treatment of a hematological malignancy. The composition comprises effective amounts of FTS or an analog thereof or a pharmaceutically acceptable salt thereof; a Bcr-Abl tyrosine kinase inhibitor; and a carrier. Methods of making the compositions are further provided.

The results of experiments described herein showed that FTS alone inhibited growth of several human leukemic and lymphoma cell lines in vitro. The results also demonstrated that in a commonly used leukemic cell line for the study of chronic myeloid-leukemia (CML) and which expresses the Bcr-Abl, oncoprotein, FTS not only inhibited leukemic cell growth but also increased the potency of imatinib. Thus, the effect of the combination was greater than additive.

### BRIEF DESCRIPTION OF THE DRAWINGS

FIG. 1A is a graph showing a dose-response curve of inhibition of growth of CEM cells (Human T-cell leukemia, ALL) at increasing concentration of FTS (μM), expressed as a percentage of control.

FIG. 1B is a graph showing a dose-response curve of inhibition of growth of HSB-2 cells (Human T-cell leukemia, ALL) at increasing concentration of FTS (μM), expressed as a percentage of control.

FIG. 1C is a graph showing a dose-response curve of inhibition of growth of 697 cells (Human T-cell leukemia, ALL) at increasing concentration of FTS (μM), expressed as a percentage of control.

FIG. 1D is a graph showing a dose-response curve of, inhibition of growth of Raji cells (Human Burkitt's lymphoma) at increasing concentration of FTS (μM), expressed as a percentage of control.

FIG. 2A is a graph showing a dose-response curve of inhibition of growth of U-937 cells (Human histiocytic lymphoma, AML) at increasing concentration of FTS (μM), expressed as a percentage of control.

FIG. 2B is a graph showing a dose-response curve of inhibition of HL-60 cells (Human, CML) at increasing concentration of FTS (μM), expressed as a percentage of control.

FIG. 2C is a graph showing a dose-response curve of inhibition of K562 cells (Human, CML) at increasing concentration of FTS (μM), expressed as a percentage of control.

FIG. 3 is a graph showing a dose-response curve of inhibition of K562 cells (Human, CML) at increasing concentrations of GLEEVEC (nM), expressed as a percentage of control.

FIG. 4A is a bar graph showing the percentage of live K562 cells (Human, CML) after treatment with FTS alone (30, 50, 70 μM); GLEEVEC alone (150, 200, 250 nM); and FTS (30, 50, 70 μM) in combination with GLEEVEC (150, 200, 250 nM).

FIG. 4B is a bar graph showing the percentage of live K562 cells (Human, CML) after treatment with FTS alone (70 μM); GLEEVEC alone (200 nM); and FTS (70 μM) in combination with GLEEVEC (200 nM) as determined by direct cell counting.

FIGS. 5 A-D are a series of four graphs illustrating the cell cycle distribution of (A) control, (B) FTS-alone (70 μM), (C) GLEEVEC-alone (STI571 200 nM), and (D) FTS (70 μM) plus GLEEVEC-treated (STI571 200 nM) K562 cells.

FIG. 6 is a graph depicting the statistical analysis of the average of two experiments of cell cycle distribution of control, FTS-alone (70 μM), GLEEVEC-alone (200 nM), and FTS (70 μM) plus GLEEVEC-treated (200 nM) K562 cells.

FIG. 7 is a graph depicting the improvement in three lineages [platelet (PLT), erythroid (HGB), and neutrophil (ANC)] over the course of FTS treatment in a 74-year-old human male subject with advanced myelodysplastic syndrome (MDS).

### DETAILED DESCRIPTION

Ras proteins act as on-off switches that regulate signal-transduction pathways controlling cell growth, differentiation, and survival. [Reuther, G. W., Der, C. J., Curr Opin Cell Biol 12:157-65 (2000)]. They are anchored to the inner leaflet of the plasma membrane, where activation of cell-surface receptors, such as receptor tyrosine kinase, induces the exchange of guanosine diphosphate (GDP) for guanosine triphosphate (GTP) on Ras and the conversion of inactive Ras-GDP to active Ras-GTP. [Scheffzek, K., Ahmadian, M. R., Kabsch, W. et al. Science 277:333-7 (1997)]. The active Ras protein promotes oncogenesis through activation of multiple Ras effectors that contribute to deregulated cell growth, differentiation, and increased survival, migration and invasion. See e.g., Downward, J., Nat. Rev. Cancer 3:11-22 (2003); Shields, J. M., et al., Trends Cell Biol 10:147-541 (2000); and Mitin, N., et al., Curr Biol 15:R563-74 (2005).

U.S. Pat. No. 5,705,528 discloses FTS and analogs thereof and their utility for treatment of solid tumors. FTS is believed to exert its antagonistic effect by dislodging activated Ras from its membrane anchor protein, thus deactivating activated Ras. See, Haklai, et al., Biochemistry 37 (5):1306-14 (1998).

FTS and its analogs are represented by formula I:

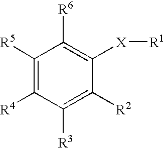

wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S.

The structure of FTS is as follows:

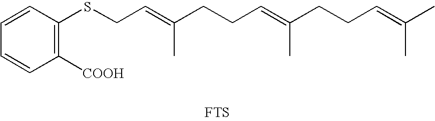

FTS analogs embraced by formula I include 5-fluoro-FTS, 5-chloro-FTS, 4-chloro-FTS, S-farnesyl-thiosalicylic acid methyl ester (FTSME), and S-geranyl,geranyl-thiosalicylic acid

(GGTS). Structures of these compounds are set forth below.

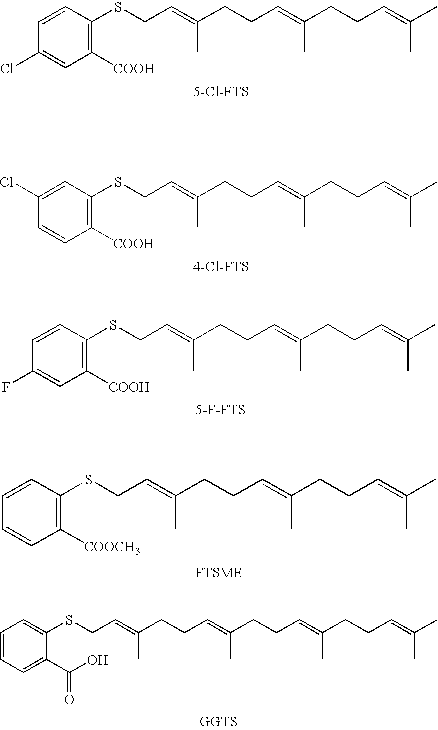

Methods for preparing the compounds of formula I are disclosed in U.S. Pat. Nos. 5,705,528 and 6,462,086. See also, Marom, M., Haklai, R., Ben-Baruch, G., Marciano, D., Egozi, Y., Kloog, Y. J Biol Chem 270:22263-70 (1995).

Pharmaceutically acceptable salts of the Ras antagonists of formula I may be useful. These salts include, for example, sodium and potassium salts. In preferred embodiments, however, FTS and its analogs are not administered in the form of a salt (i.e., they are administered in non-salified form).

In some embodiments, treatment may include administration of a Bcr-Abl tyrosine kinase inhibitor (which as used herein is inclusive of its derivatives and pharmaceutically acceptable salts). Bcr-Abl tyrosine kinase is the constitutive abnormal tyrosine kinase created by the Philadelphia chromosome abnormality found in chronic myeloid leukemia (CML). In accordance with the present invention Bcr-Abl tyrosine kinase inhibitors include, among others, imatinib [benzamide, 4-((4-methyl)-1-piperazinyl)methyl)-N-(4-methyl-3-((4-(3-pyridinyl)-2-pyrimidinyl)amino)phenyl)-]; dasatinib (N-(2-chloro-6-methylphenyl)-2-((6-(4-(2-hydroxyethyl)piperazin-1-yl)-2-methylpyrimidin-4-yl)amino)thiazole-5-carboxamide; and AMN107 [benzamide, 4-methyl-N-((3-(4-methyl-1H-imidazol-1-yl)-5-(trifluoromethyl)phenyl)-3-(4-(3-pyridinyl)-2-pyrimidinyl)amino)-].

In some embodiments, the Bcr-Abl tyrosine kinase inhibitor is imatinib, or a derivative thereof, or a pharmaceutically acceptable salt thereof. Methods of preparing and using imatinib and its derivatives and its salts as anti-cancer agents are described in U.S. Pat. Nos. 5,521,184 and 6,894,051. Imatinib (which as used herein is inclusive of its derivatives and pharmaceutically acceptable salts) is a drug approved for use in treating chronic myelogenous leukemia (CML), gastrointestinal stromal tumors (GISTs) and other malignancies. Imatinib is currently marketed as GLEEVEC® (USA) or GLIVEC® (Europe/Australia) as its mesylate salt, imatinib mesylate [4-(4-methylpiperazin-1-ylmethyl)-N-[4-methyl-3-(4-pyridin-3-yl)pyrimidin-2-ylamino)phenyl]-benzamide]. Imatinib mesylate is also referred to as CGP57148B or STI571. Other pharmaceutically acceptable salts may be selected in accordance with standard techniques as described in Berge, S. M., Bighley, L. D., and Monkhouse, D. C., J. of Pharm. Sci. 66(1):1-19 (1977). In vivo, imatinib mesylate inhibits tumor growth of Bcr-Abl transfected murine myeloid cells as well as Bcr-Abl positive leukemia lines derived from CML patients in blast crisis. [Novartis Pharma Stein AG. GLEEVEC (imatinib tablets) package insert. East Hanover, N.J.: Novartis Pharmaceuticals Corporation, November 2005].

Imatinib is also an inhibitor of the receptor tyrosine kinases for platelet-derived growth factor (PDGF) and stem cell factor (SCF), c-Kit, and inhibits PDGF- and SCF-mediated cellular events. In vitro, imatinib has been reported to inhibit proliferation and induce apoptosis in gastrointestinal stromal (GIST) cells, which express an activating c-Kit mutation. [Joensuu, H., Roberts, P. J., et al. NEJM 344(14):1052-56 (2001)].

As used herein, the term “hematological malignancy” refers to a cancer of the blood. The hematological malignancies treatable in accordance with the present invention include leukemias, lymphomas, and related disorders, including all subtypes thereof. The leukemias comprise, for example, acute myelogenous leukemia (AML), chronic myelogenous leukemia (CML), acute lymphoblastic leukemia (ALL), chronic lymphocytic leukemia (CLL), and hairy cell leukemia (HCL). The lymphomas comprise, for example, Hodgkins disease and its subtypes, as well as Non-Hodgkins lymphoma and its subtypes, including Burkitt's lymphoma. Some of the related disorders treatable in accordance with the present invention include, for example, myelodysplastic syndromes (MDS) (which can culminate in AML), myelofibrosis, myeloproliferative disease (which may evolve into MDS and AML), and amyloid due to light-chain disease.

The frequency of administration, dosage amounts, and the duration of treatment with each of the active agents may be determined depending on several factors e.g., the overall health, size and weight of the patient, the severity and type of the hematological malignancy, the patient's tolerance to the treatment, and the particular treatment regimen being administered. For example, duration of treatment with FTS or the combination of FTS and the Bcr-Abl tyrosine kinase inhibitor may last a day, a week, a year, or until remission of the disease is achieved. Thus, relative timing of administration of these active agents is not critical (e.g., FTS may be administered before, during, and after treatment with the Bcr-Abl tyrosine kinase inhibitor).

As used herein, the term “effective amount” refers to the dosages of FTS alone or in combination with the Bcr-Abl tyrosine kinase inhibitor that are effective for the treating, and thus includes dosages that ameliorate symptom(s) of the hematological malignancy or disorder, diminish extent of disease, delay or slow disease progression, or achieve partial or complete remission or prolong survival. The average daily dose of FTS generally ranges from about 50 mg to about 2000 mg, and in some embodiments, ranges from about 200 mg to about 800 mg. The average daily dose of the Bcr-Abl tyrosine kinase inhibitor generally ranges from about 50 mg to about 1,000 mg, and in some embodiments about 400 mg to about 800 mg.

In some embodiments, both drugs are administered on a daily basis, e.g., each in single once-a-day or divided doses or each in the same dosage form. They may be administered at the same or different times. In other embodiments, each drug is administered two or more times per day.

The active agents may be administered in accordance with standard methods. In preferred embodiments, FTS is administered orally. In some embodiments, FTS may be administered by dosing orally on a daily basis for three weeks, followed by a one-week “off period”, and repeating until remission is achieved. In another embodiment, FTS may be administered by dosing twice daily and continuing the treatment until remission is achieved. Parenteral administration is also suitable.

In some embodiments, the Bcr-Abl tyrosine kinase inhibitor is administered orally. In some embodiments, the Bcr-Abl tyrosine kinase inhibitor may be administered by dosing orally on a daily basis, one or more times per day, for a period of 30 months, or until remission is achieved. Parenteral administration is also suitable.

In some embodiments, the administration of FTS with the Bcr-Abl tyrosine kinase inhibitor may be cyclic. For example, in one treatment regimen, FTS (200 mg) is administered twice daily for a period of three weeks followed by a one-week, interval without FTS (“off period”) while the Bcr-Abl-tyrosine kinase inhibitor (e.g., imatinib (200 mg) is administered twice daily and continuously (e.g., without an “off period”). The treatment regimen is repeated as many times as needed, e.g., until remission is achieved. Under this regimen, imatinib is administered continuously while the FTS is administered in three-week cycles each separated by a one-week “off period”.

The treatment regimen may entail administration with FTS and imatinib continuously without interruption (i.e., without an “off period”) until remission is achieved. Some embodiments may involve administering to a patient in need thereof both actives in the same dosage form, (e.g., a capsule or a tablet) twice or thrice daily depending on the prescribed treatment schedule. For example, one schedule prescribes FTS (200 mg)/imatinib (200 mg) or FTS (200 mg)/imatinib (400 mg) twice daily in tablet form. Another schedule prescribes FTS (10 mg)/imatinib (100 mg) thrice daily in capsule form. Yet another schedule prescribes FTS (300 mg)/imatinib (300 mg) twice daily in capsule form. Alternatively, the actives are administered in separate dosage forms, (e.g., one as a capsule and the other a tablet) daily and substantially simultaneously.

Compositions for use in the present invention (which contain either or both active pharmaceutical agents) can be prepared by bringing the agent(s) into association with (e.g., mixing with) a pharmaceutically acceptable carrier. Suitable carriers are selected based in part on the mode of administration. Carriers are generally solid or liquid. In some cases, compositions may contain solid and liquid carriers. Compositions suitable for oral administration that contain either or both actives are preferably in solid dosage forms such as tablets (e.g., including film-coated, sugar-coated, controlled or sustained release), capsules, e.g., hard gelatin capsules (including controlled or sustained release) and soft gelatin capsules, powders and granules. The oral compositions, however, may be formulated in other carriers that enable administration to a patient in other oral forms, e.g., a liquid or gel. Regardless of the form, the composition is divided into individual or combined doses containing predetermined quantities of the active ingredient(s).

Oral dosage forms may be prepared by mixing the active pharmaceutical ingredient or ingredients with one or more appropriate carriers (optionally with one or more other pharmaceutically acceptable additives or excipients), and then formulating the composition into the desired dosage form e.g., compressing the composition into a tablet or filling the composition into a capsule or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethyl cellulose, sodium carboxymethyl cellulose, hydroxypropylmethyl cellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

Approved inactive ingredients for formulating oral compositions containing imatinib mesylate include: colloidal silicon dioxide, crospovidone, hydroxypropyl methylcellulose, magnesium stearate, and microcrystalline cellulose. Approved inactives for tablet coatings include: ferric oxide, yellow, hydroxypropyl methylcellulose, polyethylene glycol and talc. [Novartis Pharma Stein AG, supra.].

In an oral dosage form, the FTS is typically present in a range of about 50 mg to about 500 mg, and in some embodiments, from about 100 mg to about 300 mg. In an oral dosage form, the Bcr-Abl tyrosine kinase inhibitor e.g., imatinib, is typically present in a range of about 50 mg to about 400 mg, and in some embodiments, the amount ranges from about 100 mg to about 200 mg.

Liquid form compositions include, for example, solutions, suspensions, emulsions, syrups, elixirs and pressurized compositions. The active ingredient(s), for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, preferably in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical additives such as solubilizers, emulsifiers, buffers, preservatives, sweeteners, flavoring agents, suspending agents, thickening agents, colorants, viscosity regulators, stabilizers or osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's. Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

In order to fully illustrate the present invention and advantages thereof, the following specific examples are given, it being understood that the same is intended only as illustrative and in no way limitative.

### Example 1

The purpose of these in vitro experiments was to determine the following: (I) whether FTS alone inhibited growth of various human leukemia and lymphoma cell lines (CEM, HSB-2, 697, Raji, U-937, HL-60, and K562); and (II) whether FTS further sensitized K562 cells to imatinib.

The results of the first set of experiments, indicated that FTS inhibited growth of each of seven cell lines. Cells from lymphoblastic origin were more sensitive to FTS than cells from myelodic origin. However, these differences were small. The most sensitive cell line to FTS was Raji (Human Burkitt's lymphoma).

In the second set of experiments, the K562 cell line was chosen because it was derived using a b3-a2 fusion gene to create the Bcr-Abl chimeric oncoprotein, which is indicative of Philadelphia translocation t(9,22) found in chronic myelogenous leukemia (CML) and a subset of acute leukemias. The results indicated that in addition to inhibiting cell growth by itself, FTS increased the potency of imatinib mesylate (referred to in the following examples and drawings as “GLEEVEC”) by chemosensitizing the K562 cells to GLEEVEC. Thus, the combination of FTS and GLEEVEC resulted in an enhanced apoptotic effect on K562 cell's as determined by a FACS analysis.

### Materials and Methods

### Human Leukemia Cell Lines Examined for Sensitivity to FTS

The following cell lines were examined, representing Acute Myeloid Leukemia (AML), Acute Lymphoblastic Leukemia (ALL), and Chronic Myeloid Leukemia (CML): 1. CEM (Human T-cell leukemia, ALL) 2. HSB-2 (Human T-cell leukemia, ALL) carrying the t(1; 7) (p34; q34) involving the lck and tcrb genes and a submicroscopic del(1)(p32) leading to the Sil-Scl fusion gene 3. 697 (Human T-cell leukemia, ALL) 4. Raji (Human Burkitt's lymphoma) with overexpression and mutation in the c-Myc gene 5. U-937 (Human histiocytic lymphoma, AML) 6. HL-60 (AML) 7. K562 (Himan, CML) with a b3-a2 fusion gene to create the Bcr-Abl chimeric oncoprotein.

### Cell Growth Inhibition Assay: FTS Alone

FTS was provided by Concordia Pharmaceuticals, Inc. Cells were plated in quadruplicate at a density of 5,000 cells per ml in 24-well plates for direct cell-count assays. Cells were grown at 37° C. in RPMI containing 5% FCS, 100 μg/mL streptomycin, and 100 units/mL penicillin and maintained in a humidified atmosphere of 95% air/5% CO₂. FTS at different concentrations or the vehicle (0.1% DMSO) were added twenty-four hours (24 h) after plating and cells were counted forty-eight hours (48 h) later. Each experiment tin quadruplicate) was performed at least two times.

Impact of GLEEVEC and the Combination of GLEEVEC with FTS

First, cells were plated in quadruplicate at a density of 5,000 cells per ml in 24-well plates for direct cell-count assays. GLEEVEC at different concentrations or the vehicle (0.1% DMSO) were added at the same time.

Second, an MTS proliferation assay was performed. Cells were plated at a density of 50,000 cells per ml in 96-well plates. FTS, GLEEVEC or both drugs together added twenty-four hours (24 h) after plating at different concentrations. For the control samples, only the vehicle (0.1% DMSO) was added twenty-four hours (24 h) after plating. Forty-eight hours (48 h) later, MTS and PMS were added to the cells for one and one-half hours (1.5 h). Cell proliferation was assessed using an ELISA reader. Each experiment was performed in triplicate. Results were confirmed by direct, cell counting.

Third, a FACS analysis was performed. Cells were plated at a density of 50,000 cells per ml in 10 cm plates for fluorescence-activated cell-sorting (FACS) analysis of propidium iodide stained cells. Twenty-four hours (24 h) after plating, control samples were added with 0.1% of the vehicle DMSO, and the treatment samples were added with either 70 μM FTS or 200 nM GLEEVEC or 70 μM FTS plus 200 nM GLEEVEC. Forty-eight hours (48 h) later, propidium iodide mixed with 0.1% of, the vehicle DMSO, and the treatment samples were added with either 70 μM FTS or 200 nM GLEEVEC or 70 μM FTS plus 200 nM GLEEVEC. Forty-eight hours (48 h) later, propidium iodide mixed with 1% Triton X-100 was added to the cells, and the cell cycle distribution of the cells was assessed using a FACS machine.

### Results

### I. FTS Inhibited Leukemia Cell Growth in Human Leukemia Cell Lines

The results are set forth in Table 1. Table 1 is a chart showing inhibition of cell growth in several cell lines at increasing concentrations of FTS. The cell number in FTS-treated-cells is displayed as a percentage of controls.

TABLE 1
Inhibition of leukemia cell growth by FTS.

| | [FTS]uM | 0 | 6.25 | 12.5 | 25 | 50 | 75 | 100 |
|---|---|---|---|---|---|---|---|---|
| CEM | avg | 100% | | 89% | 78% | 71% | 32% | 12% |
| | std | 14% | | 41% | 38% | 33% | 21% | 13% |
| HSB | avg | 100% | | 88% | 53% | 38% | 22% | 4% |
| | std | 30% | | 39% | 40% | 14% | 20% | 5% |
| 697 | avg | 100% | | 27% | 44% | 34% | 4% | 2% |
| | std | 12% | | 10% | 4% | 22% | 4% | 3% |
| Raji | avg | 100% | 63% | 61% | 33% | 24% | 11% | 5% |
| | std | 12% | 4% | 12% | 15% | 7% | 6% | 3% |
| U937 | avg | 100% | | 104% | 82% | 82% | 32% | 9% |
| | std | 16% | | 15% | 20% | 30% | 29% | 12% |
| HL-60 | avg | 100% | 76% | 69% | 82% | 67% | 31% | |
| | std | 21% | 6% | 14% | 22% | 16% | 7% | |
| K562 | avg | 100% | 100% | 115% | 107% | 37% | 14% | 1% |
| | std | 14% | 26% | 34% | 44% | 14% | 12% | 3% |

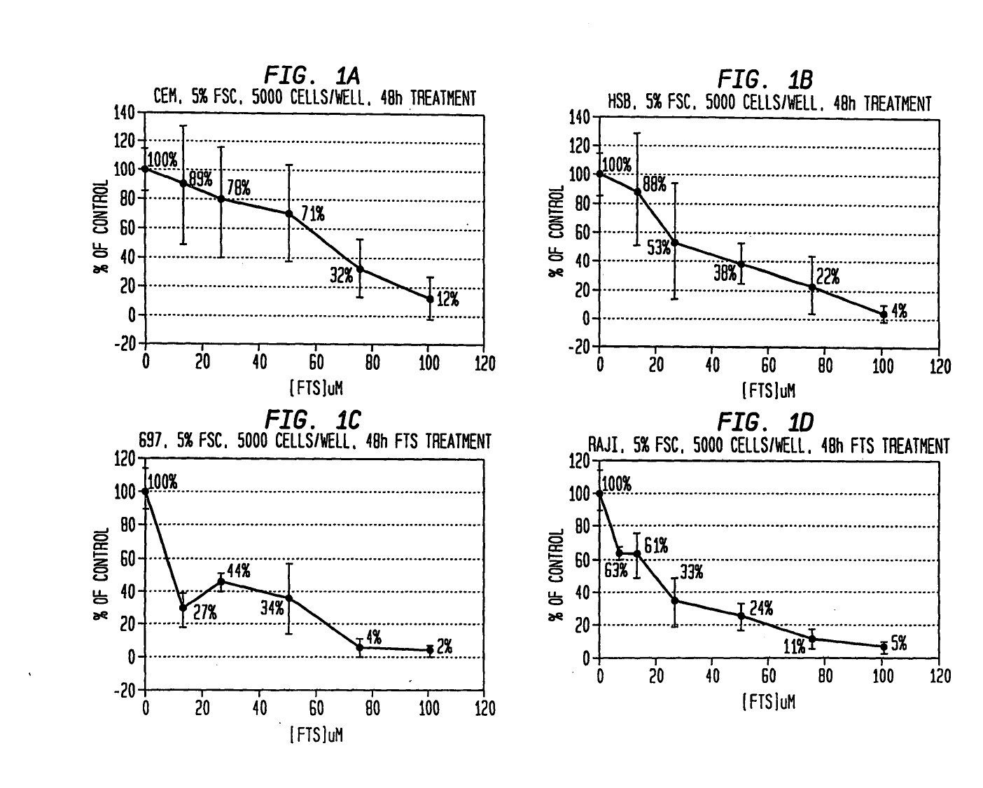

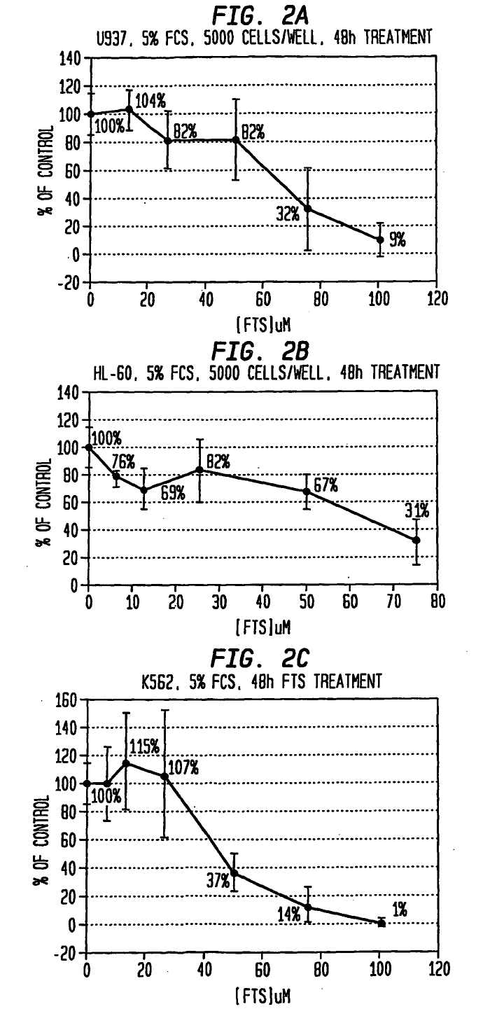

FIGS. 1 and 2 illustrate the dose response curves for each of the seven leukemia cell lines tested. FIGS. 1A-1D depict the dose response curves for cells from lymphoblastic origin. FIGS. 2A-2C depict the dose response curves for cells from myelodic origin. As shown, the cells exhibited different sensitivities to FTS. The half maximal inhibitory concentration (“IC₅₀”) values ranged from 20 μM to 70 μM. The most sensitive cell line was the Raji line. Cells from lymphoblastic origin were somewhat more sensitive than cells from myelodic origin. The difference, however, was small. (See Table 2, which shows IC₅₀ values, for inhibition of leukemia cell growth by FTS.)

TABLE 2
IC₅₀ values for FTS inhibition of leukemia cell growth

| Cell line | IC50 [uM] |
|---|---|
| CEM | 50 |
| HSB | 25 |
| 697 | 25 |
| Raji | 20 |
| U937 | 70 |
| HL-60 | 60 |
| K562 | 50 |

### II. FTS Increased the Potency of GLEEVEC(STI571) and Induced an Enhanced Apoptotic Effect in the K562 Cell Line

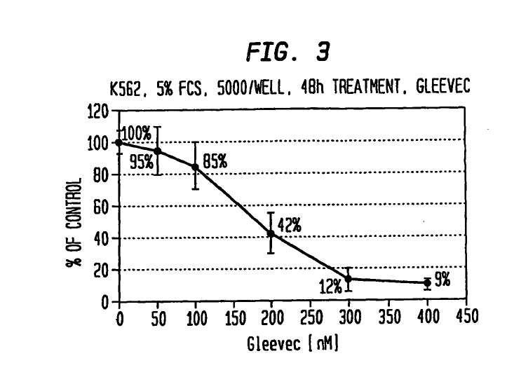

To examine the impact of the combined treatment of FTS and GLEEVEC, the K562 cells, which express the chimeric Bcr-Abl oncoprotein, were used. First, the impact of GLEEVEC alone on K562 cells was examined. As shown in FIG. 3, GLEEVEC induced a dose-dependent decrease in the number of K562 cells. The IC₅₀ was approximately 175 nM under the specified conditions.

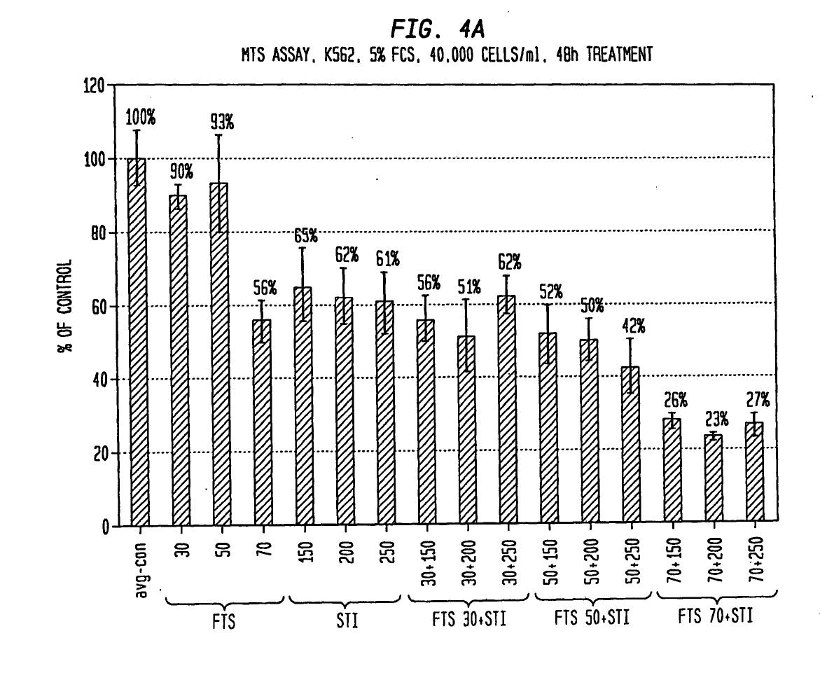

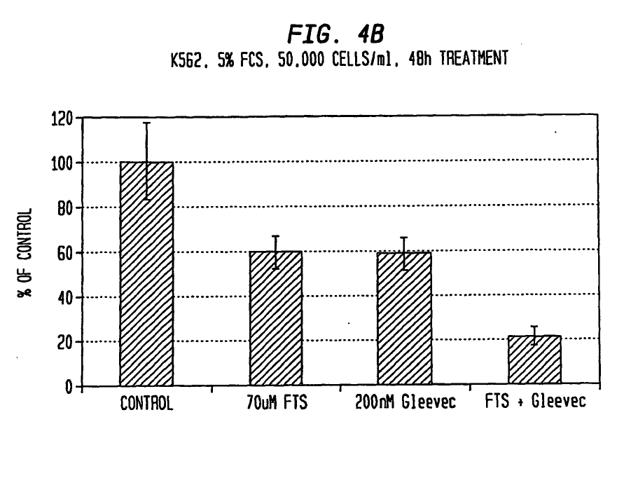

Next, an MTS proliferation assay was performed to examine the number of live cells affected by the drug combination. A four-armed-experiment was performed: Controls, GLEEVEC-treated cells, FTS-treated cells and cells treated with GLEEVEC plus FTS. Combinations of different concentrations of GLEEVEC (150, 200, 250 nM) and of different concentrations of FTS (30, 50, 70 μM) were used in these experiments. The results, as depicted in FIG. 4A and confirmed in FIG. 4B suggested that the combined treatment showed an additive growth inhibitory effect.

Next, a different analysis was performed to determine whether FTS could sensitize K562 cells to the known cytotoxic effect of GLEEVEC. [Gambacorti-Passerini, C., le Coutre, P., Mologni, L., Fanelli, M., Bertazzoli, C., Marchesi, E., Di Nicola, M., Biondi, A., Corneo, G. M., Belotti, D., Pogliani, E., Lydon, N. B., Blood Cells Mol Dis. 23(3):380-94 (1997)]. A FACS analysis of controls, GLEEVEC-treated cells (200 nM), FTS-treated cells (70 μM), and cells treated with GLEEVEC (200 nM) plus FTS (70 μM) was examined.

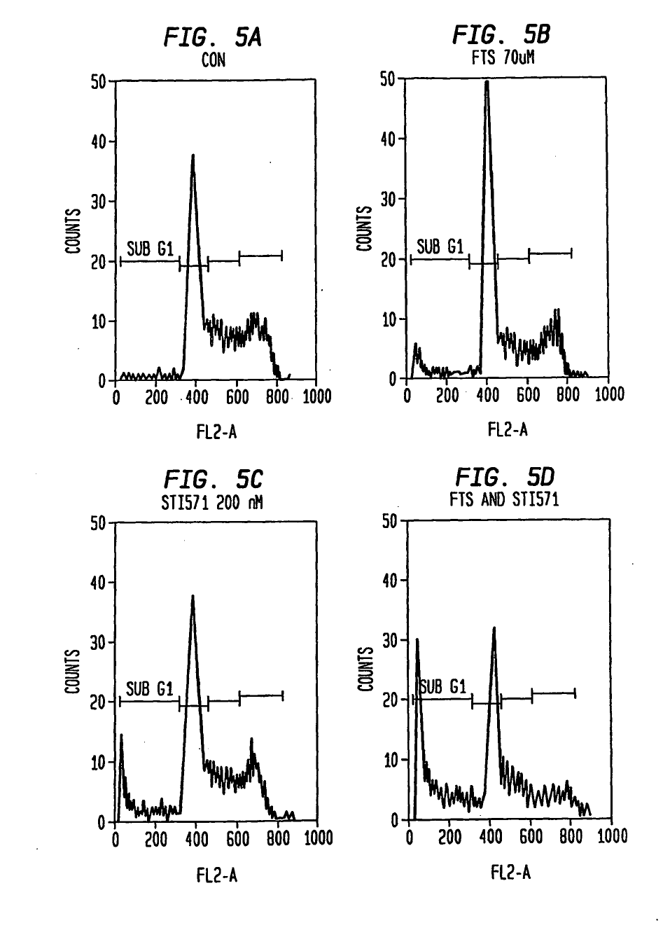

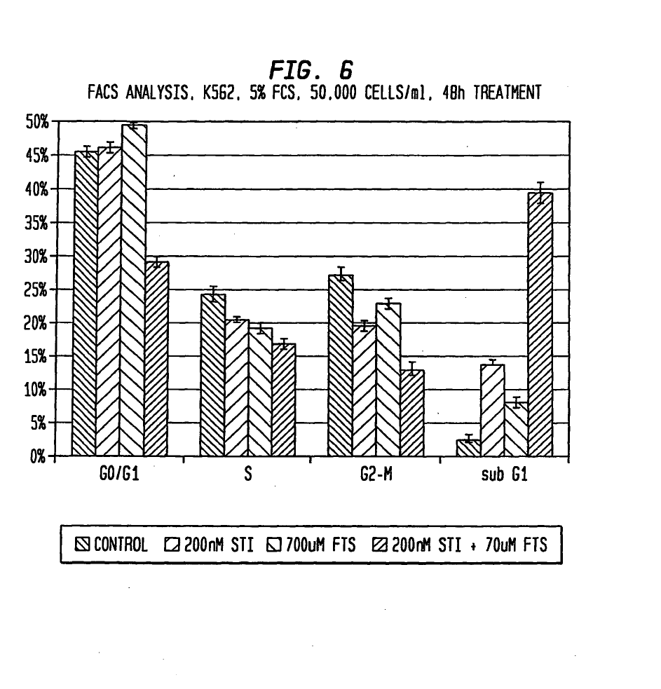

The FACS analysis showed that the combined treatment induced an enhanced apoptotic effect as indicated by the increase in the sub-G1 population of cells. The analysis is shown in FIG. 5 and the statistical analysis of two experiments is shown in FIG. 6. In the GLEEVEC-only treated cultures, 13% of the cells were in sub-G1, in the FTS-only treated cultures 8% of the cells were in sub-G1, and in the GLEEVEC plus FTS-treated cultures 39% of the cells were in sub-G1, thus revealing the enhanced apoptotic effect of the combination therapy. The results further indicated that FTS chemosensitized the K562 cells to GLEEVEC.

### Representative Formulations of Oral Dosage Forms

### Example 2

### Tablets of FTS (200 mg) and Imatinib (200 mg)

FTS (2000 g), imatinib mesylate (2400 g, equivalent to 2000 g imatinib free base), microcrystalline cellulose (2000 g), croscarmellose sodium (200 g), magnesium stearate (35 g) and colloidal silicon dioxide (15 g) are blended to uniformity and compressed into tablets weighing 665 mg. Assuming a 5% loss on material transfers and tablet press start-up, adjustment, and shut down, approximately 9,500 FTS 200 mg/imatinib 200 mg tablets are yielded.

By, adjusting tablet weight, excipient amounts, or the relative amounts of the two actives, other tablet strengths are prepared.

### Example 3

### Capsules of FTS (100 mg) and imatinib (100 mg)

FTS (2000 g), imatinib mesylate (2400 g, equivalent to 2000 g imatinib free base), microcrystalline cellulose (1000 g), croscarmellose sodium (50 g), magnesium stearate (35 g) and colloidal silicon dioxide (15 g) are blended to uniformity and filled into hard gelatin capsules. Assuming a 5% loss on material transfers and encapsulating machine start-up, adjustment, and shut down, approximately 19,000 FTS 100 mg/imatinib 100 mg capsules are yielded.

By adjusting fill weight, capsule size, excipient amounts, or the relative amounts of the two actives, other capsule strengths are prepared.

### A Phase-I Open-Label Study of S-Trans, Trans-Farnesylthiosalicylic Acid (FTS) in Human Patients with Advanced Hematologic Malignancies

### Example 4

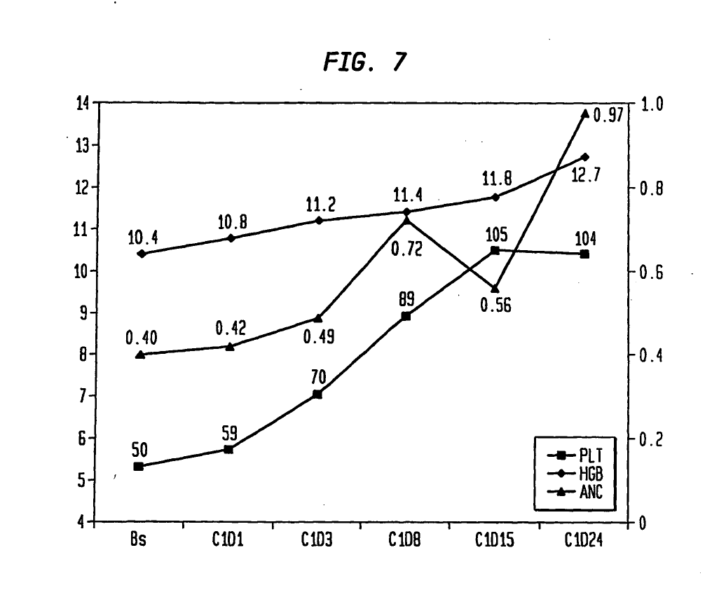

One human patient with advanced myelodysplastic syndrome (MDS) refractory to chemotherapy was treated with FTS, at a dose of 200 b.i.d. (400 mg daily), administered on days 1 to 21 of a 28 day cycle. The patient showed improvement in three lineages [platelet (PLT), erythroid (HGB), or neutrophil (ANC)] over the course of treatment. The results are graphically depicted in FIG. 7 and in Table 3 (below). Table 3 lists baseline level and best level observed.

TABLE 3
MDS patient response at 200 b.i.d. across three lineages.

| | | FTS Dose | PLT × | HGB | ANC × |
|---|---|---|---|---|---|
| Patient | Diagnosis | (mg) | 10⁹/L | g/dL | 10⁹/L |
| 74 year | MDS | 200 b.i.d. | 53 to | 10.4 to | 0.40 to |
| male | | | 105 | 12.7 | 0.97 |

### Example 5

Five human patients with advanced myelodysplastic syndrome (MDS), acute myelogenous leukemia (AML), or chronic myelogenous leukemia (CML) refractory to chemotherapy were treated with FTS at doses of 200 to 600 mg b.i.d. (400 to 1200 mg daily) administered on days 1 to 21 of a 28 day cycle. Each patient showed improvement in one lineage [platelet (PLT), erythroid (HGB), or neutrophil (ANC)] over the course of treatment. Table 4 lists baseline level and best level observed.

TABLE 4
Responses in one lineage for MDS, AML, or CML
patients treated with various doses of FTS

| | | FTS Dose | PLT × | HGB | ANC × |
|---|---|---|---|---|---|
| Patient | Diagnosis | (mg) | 10⁹/L | g/dL | 10⁹/L |
| 85 year | MDS | 200 b.i.d. | 21 to | | |
| female | | | 54 | | |
| 71 year | AML | 400 b.i.d. | | | 0.285 to |
| female | | | | | 4.503 |
| 47 year | MDS | 400 b.i.d. | | 9.7 to | |
| male | | | | 10.4 | |
| 59 year | AML | 400 b.i.d. | | | 0.144 to |
| female | | | | | 0.954 |
| 58 year | CML | 600 b.i.d. | | 9.3 to | |
| male | | | | 11.4 | |

The publications cited in the specification, patent publications and non-patent publications, are indicative of the level of skill of those skilled in the art to which this invention pertains. All these publications are herein incorporated, by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous-modifications may be made to the illustrative embodiments and that other arrangements may be devised without departing from the spirit and scope of the present invention as defined by the appended claims.

1-27. (canceled)

28. A method of treating a human having a hematological malignancy, comprising administering to the human an effective amount of FTS or an analog thereof as represented by the formula: 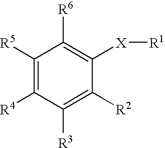
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

29. The method of claim 28, wherein the hematological malignancy is selected from the group consisting of acute myeloid leukemia, acute lymphoblastic leukemia, chronic myeloid leukemia, Burkitt's lymphoma, myelodysplastic syndrome, and a myeloproliferative disease.

30. The method of claim 28, wherein the human is administered FTS.

31. The method of claim 28, wherein the human is administered an analog of FTS which is GGTS.

32. The method of claim 28, wherein FTS or its analog is administered orally.

33. A method of treating a human having a hematological malignancy, comprising administering to a human diagnosed with a hematological malignancy effective amounts of FTS or an analog thereof as represented by the formula: 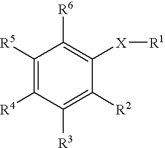
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof, and
a Bcr-Abl tyrosine kinase inhibitor.

34. The method of claim 33, wherein the hematological malignancy is selected from the group consisting of acute myeloid leukemia, acute lymphoblastic leukemia, chronic myeloid leukemia, Burkitt's lymphoma, myelodysplastic syndrome, and a myeloproliferative disease.

35. The method of claim 33, wherein the Bcr-Abl tyrosine kinase inhibitor is imatinib, or a derivative thereof, or a pharmaceutically acceptable salt thereof.

36. The method of claim 34, wherein the Bcr-Abl tyrosine kinase inhibitor is imatinib, or a derivative thereof, or a pharmaceutically acceptable salt thereof.

37. The method of claim 33, wherein the Bcr-Abl tyrosine kinase inhibitor is imatinib mesylate.

38. The method of claim 33, wherein the human is administered FTS.

39. The method of claim 33, wherein the human is administered an analog of FTS which is GGTS.

40. The method of claim 33, wherein the FTS and the Bcr-Abl tyrosine kinase inhibitor are contained in separate dosage forms.

41. The method of claim 33, wherein the FTS and the Bcr-Abl tyrosine kinase inhibitor are administered orally.

42. A pharmaceutical composition useful in the treatment of hematological malignancies, comprising effective amounts of FTS or an analog thereof as represented by the formula: 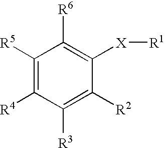
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof, and
a Bcr-Abl tyrosine kinase inhibitor, and
a pharmaceutically acceptable carrier.

43. The composition of claim 42, wherein the composition comprises FTS and imatinib mesylate.

44. The composition of claim 42, which is in the form of a tablet.

45. The composition of claim 42, which is in the form of a capsule.
